(19) United States
(12) Patent Application Publication
(10) Pub. No.: US 2008/0171000 A1
(43) Pub. Date: Jul. 17, 2008

(54) TOOTH MINERALIZATION COMPOSITIONS

(76) Inventors: Emil E. Engelman, Doylestown, PA (US); Robert J. Gambogi, Hillsborough, NJ (US); Deepak Sharma, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/972,680

(22) Filed: Jan. 11, 2008

Related U.S. Application Data
(60) Provisional application No. 60/884,735, filed on Jan. 12, 2007.

(51) Int. Cl. A61K 8/22; A61K 8/24; A61K 8/66; A61Q 11/00; A61K 8/18

(52) U.S. Cl. 424/50; 424/57; 424/49; 424/53

(57) ABSTRACT

The present invention is directed to a tooth surface mineralizing composition including a stabilized tooth mineralizing mineral composition containing calcium and phosphate and a dissociable source of a mineral that provides a mineral ionic species selected from calcium and phosphate upon dissociation.

### FIELD OF THE INVENTION

This invention relates generally to compositions that provide enhanced mineralization of tooth surfaces.

### BACKGROUND OF THE INVENTION

As people age, their teeth tend to lose surface minerals due to tooth wear and chemical erosion. This phenomenon is particularly apparent along the gum line, where tooth sensitivity may be experienced. Several products have been developed to mineralize the surface of teeth. These products typically contain calcium and phosphate containing compounds, which can deposit, help form on, and grow on the teeth through crystallization. Examples include stabilized amorphous calcium phosphate, amorphous calcium phosphate, calcium sodium phosphosilicates and hydroxyapatite. These materials may be unstable in the sense that they may agglomerate or prematurely crystallize out of solution. For these reasons, surface stabilized mineralization compositions have been developed as well as formulation strategies to stabilize these compounds. The minerals may be encapsulated in or bound to polya(mino acids) (such as poly L-lysine, poly L-glutamic acid, etc.), polypeptides or proteins, such as casein phosphopeptides, gelatin, poly electrolytes (such as Tris(hydroxymethyl) amino methane (TRIS), 2-(N-morpholino) ethanesulfonic acid (MES), and polymers. Amorphous calcium phosphate (ACP) can also be stabilized by inorganic salts such as agensium, yrophosphates, zirconium, silica and titanium. Dissociable calcium and phosphate sources can be treated in a similar manner or incompatibilities can be addressed by using non-aqueous formulation bases.

It is desirable to provide mineralization compositions that have enhanced deposition and improved efficacy. The present invention addresses this.

### SUMMARY OF THE INVENTION

The present invention is directed to improved tooth mineralizing compositions. The compositions include a stabilized tooth mineralizing mineral composition comprising calcium and phosphate and a dissociable source of a mineral that provides a mineral ionic species selected from the group consisting of calcium and phosphate upon dissociation.

### DETAILED DESCRIPTION OF THE INVENTION

The compositions of the present invention include a stabilized tooth mineralizing mineral composition. As used herein, stabilized tooth mineralizing mineral compositions means a composition that contains a source of calcium and phosphate and that has been stabilized with a proteinaceous material or a synthetic polymeric electrolyte that has an affinity for the mineral surface, resulting in a more stable form of mineral that increases the bioavailability of mineralizing ions, e.g. calcium and phosphate, in the oral cavity. Thus, the compositions are more likely to provide a mineralization benefit upon use.

Apatite, a crystalline calcium phosphate similar to the mineral found in bone, can be stabilized by collagen, gelatin, synthetic polypeptides, poly(amino acid) materials or synthetic polymeric electrolytes that have an affinity for the mineral surface. Similarly, it is known in the art that proteinaceous materials and can act as nucleation sites for crystalline materials. The proteinaceous material may be reacted in solution to encapsulate the minerals. Alternatively, the materials may be combined to surface adsorb the proteinaceous material with the minerals. Suitable sources of minerals include, but are not limited to, tricalcium phosphate, dicalcium phosphate, calcium dihydrogen phosphate, calcium pyrophosphate, hydroxyapatite, fluoroapatite, and combinations thereof. The proteinaceous material and the calcium phosphate source are typically combined at ratios of 10:1 to 0.1:10, depending on the surface area of the calcium phosphate moiety.

The amount of tooth mineralizing mineral composition in the compositions of the present invention may range from 0.1% to 10% by weight, based on the total weight of the composition.

An example of a stabilized tooth mineralizing mineral composition is a casein phosphopeptide—amorphous calcium phosphate composition. PCT Application WO 98/40406, hereby incorporated by reference, discloses stabilized tooth mineralizing mineral compositions and methods for preparing them.

The compositions of the present invention also include a dissociable source of mineral that provides a mineral ionic species selected from the group consisting of calcium and phosphate upon dissociation, that is co-delivered with the stabilized tooth mineralizing mineral composition, also known as the template material, to the tooth surface to enhance surface deposition of the mineralizing ions and subsequent growth. Dissociation of the mineral source may occur prior to or upon contact with saliva in the oral cavity. Suitable examples of dissociable mineral sources for calcium and/or phosphate include, but are not limited to, amorphous calcium phosphate, calcium phosphosilicate, calcium Na phosphosilicate, calcium glycerophosphate, calcium phosphocitrate, and independent dissociable sources of calcium, e.g. calcium chloride or other inorganic salts of calcium, and/or phosphate, e.g. Na monofluorophosphate trisodium phosphate and disodium phosphate, isolated one from the other prior to use through, e.g. multi-compartment delivery systems such as dual tube toothpaste containers, encapsulation or stabilization to prevent pre-mature crystal growth until point of use. The concentration of dissociable mineral source may be such that the concentration of mineral ionic species is greater than the solubility product constant. The level of dissociable mineral composition may range from 0.5% to 50%, or 1% to 10%, by weight, based on total weight of the composition.

The calcium:phosphate ratios will be proportional to the calcium phosphate template. Examples of suitable ratios are shown in Table 1.

TABLE 1

| Mineral | Formula | Ca/Pᵢ |
|---|---|---|
| Hydroxyapatite | Ca₁₀(PO₄)₆(OH)₂ | 1.67 |
| Calcium Pyrophosphate | Ca₂P₂O₇ | 1.00 |
| Calcium Dihydrogen Phosphate | Ca(H₂PO₄)₂ | 0.50 |
| Dicalcium Phosphate-Dihydroate | CaHPO₄₋2H2O | 1.00 |
| Tricalcium phosphate | Ca₃(PO₄)₂ | 1.50 |
| Octacalcium phosphate | Ca₈H₂(PO₄)₆•5H₂O | 1.33 |

The concentration of mineral ionic species should be greater than the solubility product constant. (i.e. I.A.P.>Ksp, for HAP ((Ca²⁺)⁵(OH⁻)(PO₄³⁻)³)>Ksp(HAP)=2.34×10⁻⁵⁹).

The compositions of the invention may be in the form of a solution, a gel, a toothpaste, a film, a lozenge, a tablet, or the like, and may include from 5% to 70% of at least one carrier. Suitable carriers include, but are not limited to, water, glycerin, propylene glycol, sorbitol, and combinations thereof Anti-caries agents, such as a fluoride agent, including, but not limited to, stannous fluoride, sodium fluoride, sodium monoflourophosphate, sodium hexafluorosilicate, and amine fluorides as well as other therapeutic agents may be useful in the compositions of the present invention at levels from 0.1% to 5% by weight, based on the total weight of the composition. The compositions of the present invention may further include binders and thickening agents including, but not limited to, colloidal silica, carrageenan, xanthan gum, methyl cellulose, carbopol, and combinations thereof, at levels ranging from 0.5% to 10% by weight, based on the total weight of the composition. Surfactants, such as sodium lauryl sulfate, betaines, sodium lauryl sarcosinate, lauryl glucoside, ethylene oxide/propylene oxide polymers and copolymers, ethoxylated sorbitans, and the like, may also be useful in the compositions of the present invention at levels ranging from 0.1% to 8% by weight, based on the total weight of the composition. The compositions of the present invention may further include from 0.1% to 7% by weight of flavors, and from 1% to 10% by weight of whiteners selected from the group consisting of hydrogen peroxide, carbamide hydrogen peroxide, enzymes from the protease, amylase and peroxidase families, pyrophosphates, sodium chlorate, organoperoxides, and inorganic peroxides.

Abrasives may be useful in the compositions of the present invention. Suitable abrasives include, but are not limited to, anhydrous dicalcium phosphate, dicalcium phosphate dihydrate, calcium carbonate, calcium pyrophosphate, sodium bicarbonate, hydrated silica, alumina, and combinations thereof. When utilized, the amount of abrasive may range from 1% to 60% by weight, based on the total weight of the composition.

The compositions of the present invention may further include a proteolytic enzyme. Examples of suitable proteolytic enzymes include, but are not limited to, serine proteases, threonine proteases, cysteine proteases, aspartic acid proteases, metalloproteases and glutamic acid proteases. Alternatively, a proteolytic enzyme may be mixed with the compositions of the present invention upon use, such as through the use of a dual chambered tube, or in compositions where ingredients do not mix until use, such as tablets and multilayered strips. Proteolytic enzymes that may be used in the present invention include papain. As used herein, papain refers to the crystalline proteolytic enzyme rather than the crude dried latex. It is a preparation from commercial dried papaya latex. According to the Merck Index, the papain molecule consists of one folded polypeptide chain of 212 residues with a molecular weight of about 23,400. If papain is used, it may be incorporated in the amount of about 0.05% to 7.5% by weight, based on total weight of the composition. Formulations will be developed to maintain the papain activity, as determined by the Milk Clot Assay Test of the Biddle-Sawyer Group. (See J. Biol. Chem., Volume 121, pages 737-745, (1937)). Traditionally papain activities of raw materials can be on the order of 800 MCU/mg. If papain having a different activity were to be used, it would be adjusted in an amount to correspond.

It is theorized that surface adsorbed proteins or peptides may control the crystal growth or integrity of calcium phosphates. Proteolytic enzymes will degrade or digest these proteins or peptides and the crystal growth properties of the calcium phosphate or the release properties of these moieties will be modified.

The activation of stabilized calcium phosphate materials may be, and in one embodiment preferably is, carried out on application to the treatment area of interest, for example the surface of teeth or bone. A calcium phosphate stabilized with an enzyme and protein, or a peptide, may be kept separate until time of activation through multicompartment delivery devices, vehicles or through encapsulation of either enzyme or stabilized calcium phosphate.

Additional ingredients that may be incorporated in the compositions of the present invention are antibacterial agents including noncationic antibacterial agents such as halogenated diphenyl ethers such as 2′,4,4′-trichloro-2-hydroxy-diphenyl ether (Triclosan) and phenolic compounds including phenols, and their homologs, mono-and polyalkyl and aromatic halophenols, resorcinol and its derivatives, bisphenolic compounds and halogenated salicylanilides. Examples of other antibacterial agents that may be included in the compositions include chlorhexidine, copper- and zinc-salts such as zinc citrate and sodium zinc citrate, sanguinarine extract, and metronidazole, quaternary ammonium compounds such as cetylpyridinium chloride, bis-guanides such as chlorhexidine digluconate, hexetidine, octenidine and alexidine. The antibacterial agent may be present in the composition in an effective antiplaque amount, typically 0.01-5% by weight based on total weight of the composition.

Anti-inflammatory agents such as ibuprofen, flurbiprofen, aspirin, indomethacin etc. may also be included in the composition. Agents useful in the treatment of dentin hypersensitivity also may be used in the present invention. Such agents include, without limitation, potassium salts such as potassium citrate, potassium chloride, amorphous calcium phosphate, potassium sulfate, potassium tartrate, oxalates and potassium nitrate.

The compositions used in the present invention may be prepared by conventional methods. Containers used to house the compositions may be of any type conventionally used, and include dual chamber products currently known and sold.

Examples are set forth below for illustrative purposes. The invention should not be construed to be limited to the details thereof.

### EXAMPLE 1

The compositions of the present invention are delivered through the use of known adhesive strip technologies. The “template” materials, e.g. hydroxyapatite, or composite hydroxyapatite, are cast or extruded to from a layer of a dry film. Onto this first layer of film, a second layer is attached via directly casting on the first layer or casting the second layer followed by lamination. The second layer will contain the dissociable calcium and phosphate sources, e.g. amorphous calcium phosphate, calcium chloride (and its equivalent), and phosphate species, e.g. trisodium phosphate, calcium sodium phosphosilicates and related compositions that will release calcium and phosphate ions on hydration. It is critical that this second layer containing the dissociable calcium and phosphate species be either extruded or cast in the absence of water. Whitening ingredients such as hydrogen peroxide and equivalents will be optionally added to this composition. The matrix of the above mentioned strip will preferably be dissolvable, however, insoluble strips are also conceivable.

### EXAMPLE 2

Dentifrice compositions are prepared by combining the materials in Table 1 in the appropriate mixing vessels and combining in the order typical for dentifrice compositions. Combinations of these compositions will provide enhanced surface mineralization compared to individual formulations. Combinations illustrative of the invention are as follows: Formula A+B, A+C, B+D, and A+E. Formulation F represents an example where the non-aqueous nature of the formulation is leveraged for free calcium phosphate source in lieu of a multi compartment system.

TABLE 1

| Ingredient | Formula A | Formula B | Formula C | Formula D | Formula E | Formula F |
|---|---|---|---|---|---|---|
| USP Sorbitol Solution 70% | 45.000 | 45.000 | 45.000 | 45.000 | | |
| Glycerin, USP | 10.000 | 10.000 | 10.000 | 10.000 | 71.14 | 70.14 |
| Water | 19.840 | 19.840 | 20.640 | 21.157 | | |
| Hydrated Silica Abrasive | 17.000 | 17.000 | 17.000 | 17.000 | 17 | 17 |
| Flavor | 1.200 | 1.200 | 1.200 | 1.200 | 1.2 | 1.2 |
| Sodium Lauryl Sulfate | 1.000 | 1.000 | 1.000 | 1.000 | 1.000 | 1.000 |
| Papain | 0.800 | 0.800 | | | | |
| Carbopol | 0.500 | 0.500 | 0.500 | 0.500 | 0.5 | 0.5 |
| Xanthan Gum | 0.500 | 0.500 | 0.500 | 0.500 | | |
| Titanium Dioxide | 0.500 | 0.500 | 0.500 | 0.500 | 0.5 | 0.5 |
| Carboxymethylcellulose | 0.500 | 0.500 | 0.500 | 0.500 | | |
| Sweetner | 0.400 | 0.400 | 0.400 | 0.400 | 0.400 | 0.400 |
| Na Monofluorophosphate | 0.760 | 0.760 | 0.760 | | 0.76 | 0.76 |
| Sodium Fluoride | | | | 0.243 | | |
| Hydroxyapatite | 1.000 | 1.000 | | | | |
| Protein-HAP Complex | | | | 1.000 | | 1.000 |
| Peptide Stabilized ACP | | | 1.000 | | | |
| Calcium Chloride | | 1.000 | | | | |
| Disodium Phosphate | 1.000 | | 1.000 | 1.000 | | |
| Calcium Na Phosphosilicate | | | | | 7.5 | 7.5 |

HAP = hydroxyapatite
ACP = amorphous calcium phosphate

### EXAMPLE 3

Below is a strip example where a water-soluble calcium phosphate source (in this case calcium sodium phosphosilicate) is in one layer and the mineral stabilized HAP is in another layer. If processed under non-aqueous conditions, such as Formula F above, it is possible to have a water-soluble calcium phosphate source combined with mineral in a single layer. On hydration in the oral cavity this combination will work together to promote surface mineral on the tooth. Dissolvable variants are feasible and included in this invention.

| Inner Layer | | Outer Layer | |
|---|---|---|---|
| Ingredients | w/w (%) | Ingredients | w/w (%) |
| Ethanol (200 | 64.87 | Ethanol −95% (190 | 69.2 |
| proof) | | proof) | |
| Peroxydone K-90 | 31.43 | PEG 1000 | 1.2 |
| PEG-4500 | 0.5 | DI Water | 10 |
| Stabilized Apatite | 3 | SODIUM SACCHARIN | 0.5 |
| | | (S) POWDER FCC, | |
| | | USP | |
| L-MENTHOL | 0.1 | HPMCP HP-55S | 18 |
| CRYSTALS | | | |
| SODIUM | 0.1 | Frosty White Flavor | 1 |
| SACCHARIN (S) | | (AN147634) | |
| POWDER FCC, | | | |
| USP | | | |
| | 100 | Calcium Na | 0.1 |
| | | Phosphosilicate | |

We claim:

1. A tooth surface mineralizing composition, comprising:
a stabilized tooth mineralizing mineral composition; and
a dissociable source of mineral which provides a mineral ionic species selected from the group consisting of calcium and phosphate upon dissociation.

2. The tooth surface mineralizing composition according to claim 1 comprising from 0.1 to 10 percent by weight of said stabilized tooth mineralizing mineral composition.

3. The tooth surface mineralizing composition according to claim 2 comprising from 0.5% to 50% by weight of said dissociable source of mineral.

4. The tooth surface mineralizing composition according to claim 2 wherein said stabilized tooth mineralizing mineral composition is selected from the group consisting of a casein phosphopeptide amorphous calcium phosphate and stabilized hydroxyapatite.

5. The tooth surface mineralizing composition according to claim 4 wherein the amount of dissociable mineral composition is such that the concentration of mineral ionic species is greater than the solubility product constant.

6. The tooth surface mineralizing composition according to claim 5 wherein said source of dissociable mineral is selected from the group consisting of amorphous calcium phosphate, calcium phosphosilicate, calcium Na phosphosilicate, calcium glycerophosphate, calcium phosphocitrate, calcium chloride and other inorganic salts of calcium, Na monofluorophosphate, trisodium phosphate and disodium phosphate.

7. The tooth surface mineralizing composition according to claim 6 wherein the amount of dissociable mineral composition is such that the concentration of mineral ionic species is greater than the solubility product constant.

6. The tooth surface mineralizing composition according to claim 2 wherein said tooth mineralizing mineral is protease-resistant.

7. The tooth surface mineralizing composition according to claim 1 further comprising a proteolytic enzyme.

8. The tooth surface mineralizing composition according to claim 6 comprising from about 0.05 to 7.5 percent by weight of a proteolytic enzyme selected from the group consisting of serine proteases, threonine proteases, cysteine proteases, aspartic acid proteases, metalloproteases and glutamic acid proteases.

9. The tooth surface mineralizing composition according to claim 1 further comprising an abrasive.

10. The tooth surface mineralizing composition according to claim 1 further comprising a whitener selected from the group consisting of hydrogen peroxide, carbamide hydrogen peroxide, protease enzymes, polyphosphates and pyrophosphates.
